(19) United States
(12) Patent Application Publication
(10) Pub. No.: US 2025/0061368 A1
(43) Pub. Date: Feb. 20, 2025

(54) COMPUTER-READABLE RECORDING MEDIUM STORING INFORMATION PROCESSING PROGRAM, INFORMATION PROCESSING METHOD, AND INFORMATION PROCESSING APPARATUS

(71) Applicant: Fujitsu Limited, Kawasaki-shi, Kanagawa (JP)

(72) Inventors: Naoki IIJIMA, Kawasaki (JP); Satoshi IMAMURA, Kawasaki (JP)

(73) Assignee: Fujitsu Limited, Kawasaki-shi, Kanagawa (JP)

(21) Appl. No.: 18/753,055

(22) Filed: Jun. 25, 2024

(30) Foreign Application Priority Data
Aug. 14, 2023 (JP) 2023-132185

(51) Int. Cl. G06N 10/60; G16C 20/20

(52) U.S. Cl. CPC G06N 10/60; G16C 20/20

(57) ABSTRACT

A recording medium stores a program for executing processing of: obtaining a first value of each first parameter calculated first by the VQE that calculates values of parameter of a quantum circuit and a second value of each first parameters calculated last by the VQE when an active space of a target molecule is reduced; obtaining a third value of each second parameter calculated first by the VQE such that the parameters are calculated once, when the active space of the target molecule is not reduced; specifying one first parameter corresponding to the first value closest to the third value of the second parameter to associate the specified first parameter with the second parameter; and when the active space of the target molecule is not reduced, setting an initial value of each second parameter to the second value of one first parameter specified to be associated with each second parameter.

### CROSS-REFERENCE TO RELATED APPLICATION

This application is based upon and claims the benefit of priority of the prior Japanese Patent Application No. 2023-132185, filed on Aug. 14, 2023, the entire contents of which are incorporated herein by reference.

### FIELD

The embodiment discussed herein is related to a computer-readable recording medium storing an information processing program, an information processing method, and an information processing apparatus.

### BACKGROUND

There has been a variational quantum eigensolver (VQE) that is used in calculation of ground energy of a target molecule in fields such as material development and drug discovery research. For example, the VQE repeatedly executes a series of processes of updating parameters of a quantum circuit, executing the quantum circuit, and calculating an expected value of the ground energy.

Japanese Laid-open Patent Publication No. 2004-118658 is an example of related art.

### SUMMARY

According to an aspect of the embodiments, a non-transitory computer-readable recording medium stores an information processing program that causes a computer to execute processing of: obtaining a first value of each of a plurality of first parameters calculated first by the variational quantum eigensolver that recurrently calculates values of parameters of a quantum circuit and a second value of each of the plurality of first parameters calculated last by the variational quantum eigensolver by executing the variational quantum eigensolver for a case where an active space of a target molecule is reduced; obtaining a third value of each of a plurality of second parameters calculated first by the variational quantum eigensolver by executing the variational quantum eigensolver such that the parameters of the quantum circuit are calculated once, for a case where the active space of the target molecule is not reduced; specifying, for each of the plurality of second parameters, one of the first parameters that corresponds to the first value closest to the third value of the second parameter, based on the calculated first value and the calculated third value, to associate the specified first parameter with the second parameter; and when the variational quantum eigensolver is to be executed for the case where the active space of the target molecule is not reduced, setting an initial value of each of the plurality of second parameters to the second value of the one of the plurality of first parameters specified to be associated with the each of the plurality of second parameters.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is an explanatory diagram illustrating an example of an information processing method according to an embodiment;

FIG. 2 is an explanatory diagram illustrating an example of an information processing system;

FIG. 3 is a block diagram illustrating a hardware configuration example of an information processing apparatus;

FIG. 4 is a block diagram illustrating a functional configuration example of the information processing apparatus;

FIG. 5 is an explanatory diagram (part 1) illustrating an operation example of the information processing apparatus;

FIG. 6 is an explanatory diagram (part 2) illustrating the operation example of the information processing apparatus;

FIG. 7 is an explanatory diagram (part 3) illustrating the operation example of the information processing apparatus;

FIG. 8 is a flowchart (part 1) illustrating an example of an overall processing procedure;

FIG. 9 is a flowchart (part 2) illustrating the example of the overall processing procedure; and

FIG. 10 is a flowchart illustrating an example of a calculation processing procedure.

### DESCRIPTION OF EMBODIMENTS

For example, there is a technique in which function approximation is updated by updating learning parameters of each of local models such that a predetermined error index is minimized independently for each local model.

However, the related art has a problem of an increase of processing time taken to execute the VQE. For example, the processing time taken to execute the VQE increases unless initial values of the parameters of the quantum circuit are set to appropriate values.

According to one aspect, an object of the present disclosure is to reduce the processing time taken to execute the VQE.

Hereinafter, an embodiment of an information processing program, an information processing method, and an information processing apparatus according to the present disclosure will be explained in detail with reference to the drawings.

### One Example of Information Processing Method According to Embodiment

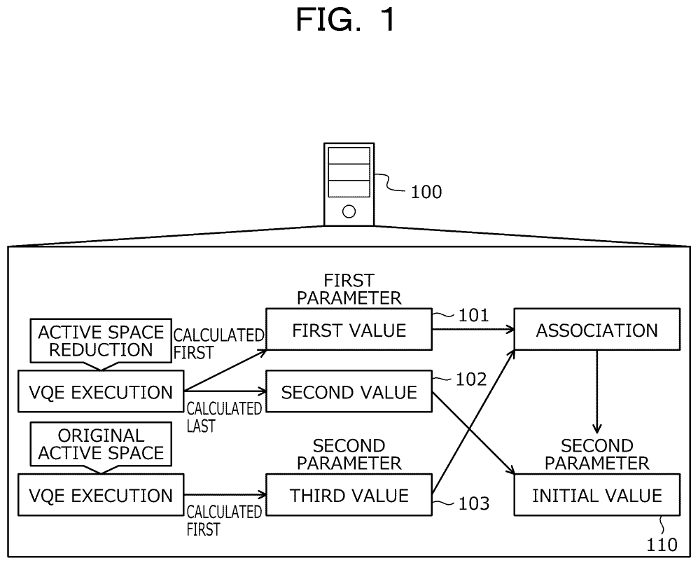

FIG. 1 is an explanatory diagram illustrating one example of the information processing method according to the embodiment. An information processing apparatus 100 is a computer that executes a VQE. The VQE is also referred to as, for example, variational quantum eigensolver. The information processing apparatus 100 is, for example, a server, a personal computer (PC), or the like.

In fields such as material development and drug discovery research, there is a demand for calculation of ground energy of a target molecule to analyze properties of the target molecule. In a related art, the VQE is sometimes used in calculation of the ground energy of the target molecule.

The VQE is a hybrid algorithm of quantum computation and classical computation in which an eigenvalue is obtained by calculus of variations. For example, the VQE performs classical computation of initializing a quantum state, and then repeatedly performs a series of processes. For example, the series of processes is performing classical computation of setting parameters of a quantum circuit, performing quantum computation of executing the quantum circuit, performing classical computation of calculating an expected value of the ground energy, and performing classical computation of calculating the parameters of the quantum circuit. The classical computation of calculating the parameters of the quantum circuit uses, for example, a gradient method.

In the classical computation of setting the parameters of the quantum circuit, the parameters of the quantum circuit are set to initial values specified in advance by a user in, for example, the first series of processes. In the classical computation of setting the parameters of the quantum circuit, the parameters of the quantum circuit are set to values calculated in the classical computation of calculating the parameters of the quantum circuit in, for example, the second series of processes and beyond. For example, the VQE repeatedly performs the series of processes until the ground energy satisfies a convergence condition. The convergence condition is, for example, such a condition that an amount of change between a previously-calculated ground energy and a currently-calculated ground energy becomes less than a threshold. For example, the convergence condition may be such a condition that the ground energy becomes equal to or less than a threshold.

However, the related art has a problem of an increase of processing time taken to execute the VQE. For example, when the initial values of the parameters of the quantum circuit are not set to appropriate values, time taken for the ground energy to satisfy the convergence condition increases, and the processing time taken to execute the VQE increases. It tends to be difficult for the user to specify appropriate values to be set as the initial values of parameters of the quantum circuit.

In this respect, a method is conceivable in which values of the parameters of the quantum circuit obtained in past in solving of a problem for calculating the ground energy of the target molecule of a second interatomic distance is used as the initial values of the parameters of the quantum circuit in solving of a problem for calculating the ground energy of the target molecule of a first interatomic distance. In this method, before solving the problem for calculating the ground energy of the target molecule, another problem of the same scale as the problem for calculating the ground energy of the target molecule is solved, and there is a problem that the time taken to complete execution of the VQE to calculate the ground energy of the target molecule increases.

In the present embodiment, explanation is given of an information processing method capable of reducing the processing time taken to execute the VQE. For example, this information processing method may reduce the processing time taken to execute the VQE by using an active space of the target molecule to set the initial values of the parameters of the quantum circuit to appropriate values.

The active space defines an electron configuration of a molecule. For example, the active space expresses an orbital for which the electron configuration is to be examined among orbitals of the molecule. The larger the number of orbitals for which the electron configuration is to be examined is, the longer the processing time taken to execute the VQE for the molecule tends to be. Reducing the active space means reducing the number of orbitals for which the electron configuration is to be examined. For example, in the VQE, when the number of orbitals for which the electron configuration is to be examined is reduced by one, the number of qubits is reduced by two.

In FIG. 1, the target molecule is present. The information processing apparatus 100 includes the VQE. As described above, the VQE recurrently calculates the values of the parameters of the quantum circuit. Assume that the information processing apparatus 100 obtains a request demanding execution of the VQE for the target molecule. For example, the request demands calculation of the ground energy of the target molecule.

(1-1) The information processing apparatus 100 executes the VQE for the case where the active space of the target molecule is reduced. As a result of executing the VQE, the information processing apparatus 100 obtains a first value 101 of each of multiple first parameters calculated first by the VQE and a second value 102 of each of the multiple first parameters calculated last by the VQE. The first parameters are the parameters of the quantum circuit in the case where the active space of the target molecule is reduced.

The information processing apparatus 100 may thus obtain a guideline for setting an initial value 110 of each of multiple second parameters used when the VQE is executed for the case where the active space of the target molecule is not reduced. The second parameters are the parameters of the quantum circuit in the case where the active space of the target molecule is not reduced. The number of second parameters is preferably larger than the number of first parameters. There may be a case where the number of second parameters is the same as the number of first parameters. The information processing apparatus 100 may reduce time taken to obtain the guideline for setting the initial value 110 of each of the multiple second parameters, from that in the case where the VQE is executed for the case where an active space of a target molecule with a different interatomic distance from that of the current target molecule is not reduced.

(1-2) The information processing apparatus 100 executes the VQE such that the parameters of the quantum circuit are calculated once, for the case where the active space of the target molecule is not reduced. For example, the information processing apparatus 100 executes the VQE such that the parameters of the quantum circuit are calculated only once, for the case where the active space of the target molecule is not reduced. As a result of executing the VQE, the information processing apparatus 100 obtains a third value 103 of each of the multiple second parameters calculated first by the VQE. The information processing apparatus 100 may thus obtain the guideline for setting the initial value 110 of each of the multiple second parameters used when the VQE is continued hereinafter for the case where the active space of the target molecule is not reduced.

(1-3) The information processing apparatus 100 specifies one of the multiple first parameters that corresponds to each of the multiple second parameters, based on the calculated first value 101 and the calculated third value 103. For example, the information processing apparatus 100 specifies, for each of the multiple second parameters, one of the multiple first parameters for which the first value 101 closest to the third value 103 of the second parameter is calculated, to associate the specified first parameter with the second parameter. The information processing apparatus 100 may thereby specify the first parameter similar to the second parameter, and may specify the first parameter preferable to be referred to in setting of the initial value 110 of the second parameter.

(1-4) The information processing apparatus 100 sets the initial value 110 of each of the multiple second parameters used when the VQE is executed for the case where the active space of the target molecule is not reduced. For example, the information processing apparatus 100 sets the initial value 110 of each of the multiple second parameters to the second value 102 of one of the multiple first parameters specified to be associated with the second parameter. The information processing apparatus 100 may thereby set the initial value 110 of each of the multiple second parameters to an appropriate value. The information processing apparatus 100 may thus reduce the processing time taken to execute the VQE for the case where the active space of the target molecule is not reduced.

For example, as preparation of setting the initial value 110 of each of the multiple second parameters, the information processing apparatus 100 may execute the VQE for the case where the active space of the target molecule is reduced. For example, as the preparation of setting the initial value 110 of each of the multiple second parameters, the information processing apparatus 100 may execute the VQE such that the parameters of the quantum circuit are calculated once, for the case where the active space of the target molecule is not reduced.

Accordingly, for example, the information processing apparatus 100 may reduce the time taken to obtain the guideline for setting the initial value 110 of each of the multiple second parameters. For example, the information processing apparatus 100 may then execute the VQE for the case where the active space of the target molecule is not reduced, based on the appropriate initial value 110 of each of the multiple second parameters, and may reduce the processing time taken to execute the VQE.

(1-5) The information processing apparatus 100 may execute the VQE for the case where the active space of the target molecule is not reduced, based on the set initial value 110 of each of the multiple second parameters. For example, as a result of executing the VQE, the information processing apparatus 100 calculates the expected value of the ground energy of the target molecule. For example, the information processing apparatus 100 outputs the expected value of the ground energy of the target molecule such that it is possible for the user to refer to the expected value. The information processing apparatus 100 may thereby facilitate analysis of the properties and the like of the target molecule by the user.

Although the case where the information processing apparatus 100 executes the VQE such that the parameters of the quantum circuit are calculated only once for the case where the active space of the target molecule is not reduced has been explained, the embodiment is not limited to this case. For example, there may be a case where the information processing apparatus 100 executes the VQE such that the parameters of the quantum circuit are calculated twice or more for the case where the active space of the target molecule is not reduced. Meanwhile, reduction of the processing amount may be achieved in the case where the information processing apparatus 100 executes the VQE such that the parameters of the quantum circuit are calculated only once, compared to, for example, the case where the information processing apparatus 100 executes the VQE such that the parameters of the quantum circuit are calculated twice or more.

Although the case where the information processing apparatus 100 compares the first value 101 of each of the multiple first parameters calculated first by the VQE with the third value 103 of each of the multiple second parameters calculated first by the VQE has been explained, the embodiment is not limited to this case. For example, there may be a case where the information processing apparatus 100 compares a fourth value of each of the multiple first parameters calculated in a first number of calculation by the VQE with a fifth value of each of the multiple second parameters calculated in a second number of calculation by the VQE.

For example, the first number of calculation and the second number of calculation are the same value. For example, the first number of calculation is preferably a value smaller than the number of last calculation of the value of each of the multiple first parameters by the VQE. For example, the second number of calculation is preferably a value smaller than the number of last calculation of the value of each of the multiple second parameters by the VQE. For example, the information processing apparatus 100 compares the fourth value of each of the multiple first parameters calculated at a predetermined number of calculation by the VQE with the fifth value of each of the multiple second parameters calculated first by the VQE, and specifies the first parameter corresponding to the second parameter.

Although the case where the function as the information processing apparatus 100 is implemented by a single computer has been explained, the embodiment is not limited to this case. For example, there may be a case where the function as the information processing apparatus 100 is implemented by cooperation of multiple computers. For example, there may be a case where the function as the information processing apparatus 100 is implemented on a cloud.

### Example of Information Processing System 200

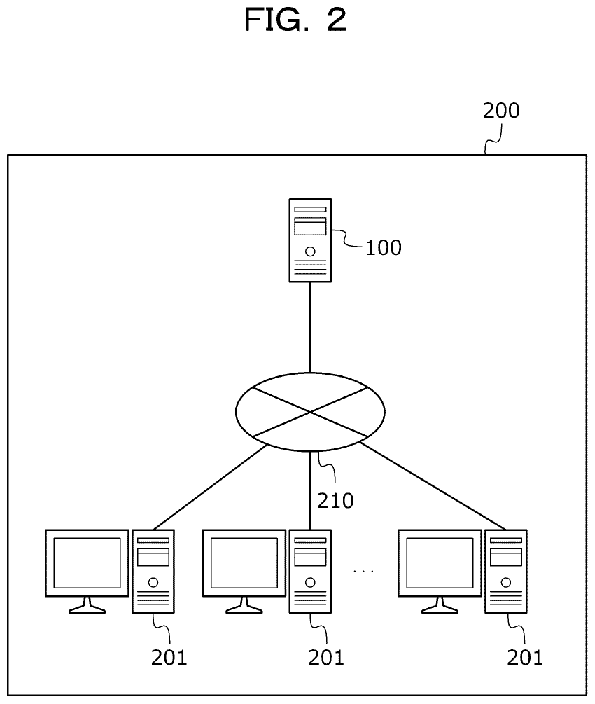

Next, an example of an information processing system 200 to which the information processing apparatus 100 illustrated in FIG. 1 is applied will be explained by using FIG. 2.

FIG. 2 is an explanatory diagram illustrating an example of the information processing system 200. In FIG. 2, the information processing system 200 includes the information processing apparatus 100 and client apparatuses 201.

In the information processing system 200, the information processing apparatuses 100 and the client apparatus 201 are coupled to each other via a wired or wireless network 210. The network 210 is, for example, a local area network (LAN), a wide area network (WAN), the Internet, or the like.

The information processing apparatus 100 is a computer for executing the VQE. The information processing apparatus 100 receives a processing request demanding calculation of the expected value of the ground energy of the target molecule, from the client apparatuses 201. The processing request includes, for example, information specifying the target molecule.

The information processing apparatus 100 executes the VQE for the case where the active space of the target molecule is reduced, in response to the processing request. As a result of executing the VQE, the information processing apparatus 100 obtains the first value of each of the multiple first parameters calculated first by the VQE and the second value of each of the multiple first parameters calculated last by the VQE. The information processing apparatus 100 executes the VQE such that the parameters of the quantum circuit are calculated once, for the case where the active space of the target molecule is not reduced. As a result of executing the VQE, the information processing apparatus 100 obtains the third value of each of the multiple second parameters calculated first by the VQE.

The information processing apparatus 100 specifies, for each of the multiple second parameters, one of the multiple first parameters for which the first value closest to the third value of the second parameter is calculated, based on the calculated first value and the calculated third value, to associate the specified first parameter with the second parameter. The information processing apparatus 100 sets the initial value of each of the multiple second parameters used when the VQE is executed for the case where the active space of the target molecule is not reduced. For example, the information processing apparatus 100 sets the initial value of each of the multiple second parameters to the second value of one of the multiple first parameters specified to be associated with the second parameter.

The information processing apparatus 100 executes the VQE for the case where the active space of the target molecule is not reduced, based on the set initial value of each of the multiple second parameters. For example, as a result of executing the VQE, the information processing apparatus 100 calculates the expected value of the ground energy of the target molecule. For example, the information processing apparatus 100 transmits the expected value of the ground energy of the target molecule to the client apparatuses 201. For example, the information processing apparatus 100 is a server, a PC, or the like.

Each client apparatus 201 is a computer used by an analyst who analyzes the properties and the like of the target molecule. The client apparatus 201 generates the processing request demanding calculation of the expected value of the ground energy of the target molecule based on an operation input by the analyst, and transmits the processing request to the information processing apparatus 100. The client apparatus 201 receives the expected value of the ground energy of the target molecule from the information processing apparatus 100. The client apparatus 201 outputs the expected value of the ground energy of the target molecule such that it is possible for the analyst to refer to the expected value. For example, the client apparatus 201 is a PC, a tablet terminal, a smartphone, or the like.

Although the case where the information processing apparatus 100 is a computer different from the client apparatuses 201 has been explained, the embodiment is not limited to this case. For example, there may be a case where the information processing apparatus 100 has the function as the client apparatus 201 and operates also as the client apparatus 201.

### Hardware Configuration Example of Information Processing Apparatus 100

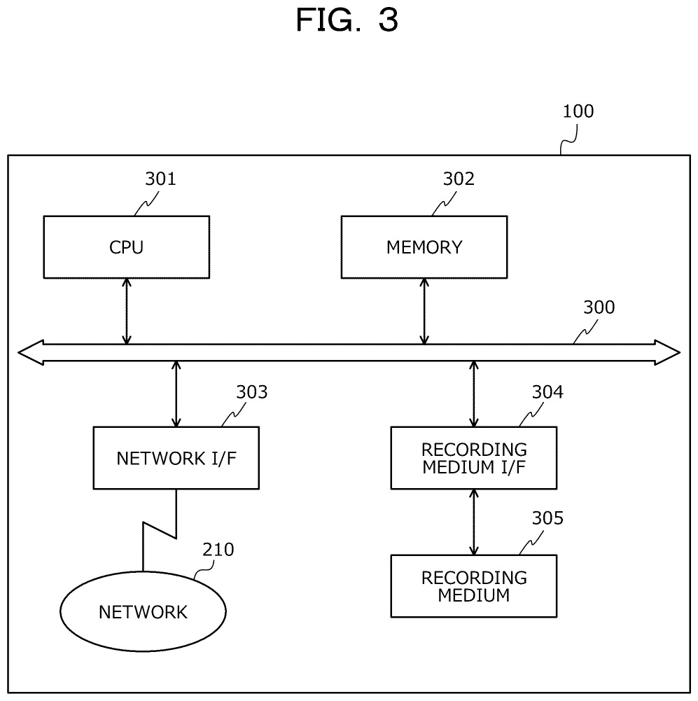

Next, a hardware configuration example of the information processing apparatus 100 will be explained by using FIG. 3.

FIG. 3 is a block diagram illustrating the hardware configuration example of the information processing apparatus 100. In FIG. 3, the information processing apparatus 100 includes a central processing unit (CPU) 301, a memory 302, a network interface (I/F) 303, a recording medium I/F 304, and a recording medium 305. The components are coupled to one another by a bus 300.

The CPU 301 is responsible for control of the entire information processing apparatus 100. For example, the memory 302 includes a read-only memory (ROM), a random-access memory (RAM), a flash ROM, and the like. For example, the flash ROM and the ROM store various programs, and the RAM is used as a work area of the CPU 301. The programs stored in the memory 302 are loaded by the CPU 301 to cause the CPU 301 execute coded processing.

The network I/F 303 is coupled to the network 210 through a communication line, and is coupled to other computers via the network 210. The network I/F 303 serves as an interface between the network 210 and the inside of the information processing apparatus 100, and controls input and output of data from and to the other computers. For example, the network I/F 303 is a modem, a LAN adapter, or the like.

The recording medium I/F 304 controls reading and writing of data from and to the recording medium 305 according to control of the CPU 301. For example, the recording medium I/F 304 is a disk drive, a solid-state drive (SSD), a Universal Serial Bus (USB) port, or the like. The recording medium 305 is a non-volatile memory that stores data written under control of the recording medium I/F 304. For example, the recording medium 305 is a disk, a semiconductor memory, a USB memory, or the like. The recording medium 305 may be removably attached to the information processing apparatus 100.

In addition to the components described above, the information processing apparatus 100 may include, for example, a keyboard, a mouse, a display, a printer, a scanner, a microphone, a speaker, and the like. The information processing apparatus 100 may include multiple recording medium I/Fs 304 and multiple recording media 305. The information processing apparatus 100 does not have to include the recording medium I/F 304 or the recording medium 305.

### Hardware Configuration Example of Client Apparatuses 201

Since a hardware configuration example of the client apparatuses 201 is similar to, for example, the hardware configuration example of the information processing apparatus 100 illustrated in FIG. 3, explanation thereof will be omitted.

### Functional Configuration Example of Information Processing Apparatus 100

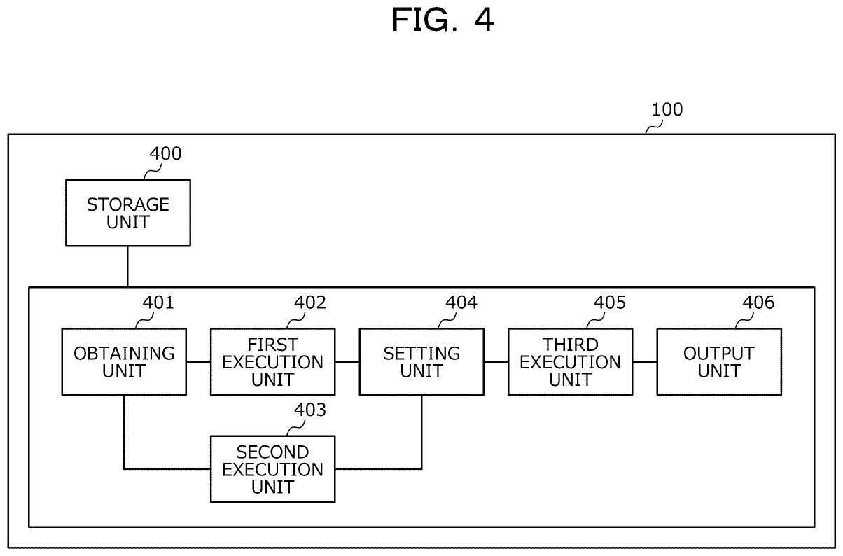

Next, a functional configuration example of the information processing apparatus 100 will be explained by using FIG. 4.

FIG. 4 is a block diagram illustrating the functional configuration example of the information processing apparatus 100. In FIG. 4, the information processing apparatus 100 includes a storage unit 400, an obtaining unit 401, a first execution unit 402, a second execution unit 403, a setting unit 404, a third execution unit 405, and an output unit 406.

For example, the storage unit 400 is implemented by storage areas of the memory 302, the recording medium 305, and the like illustrated in FIG. 3. Although the case where the storage unit 400 is included in the information processing apparatus 100 will be explained below, the embodiment is not limited to this case. For example, there may be a case where the storage unit 400 is included in an apparatus different from the information processing apparatus 100 and reference of contents stored in the storage unit 400 is possible from the information processing apparatus 100.

The obtaining unit 401 to the output unit 406 function as an example of a control unit. For example, the functions of the obtaining unit 401 to the output unit 406 are implemented by, for example, causing the CPU 301 to execute programs stored in the storage areas of the memory 302, the recording medium 305, and the like illustrated in FIG. 3, or by using the network I/F 303. For example, a processing result of each functional unit is stored in the storage area of the memory 302, the recording medium 305, or the like illustrated in FIG. 3.

The storage unit 400 stores various types of information to be referred to or updated in processing of each functional unit. For example, the storage unit 400 stores the VQE. For example, the VQE recurrently calculates the values of the parameters of the quantum circuit related to the target molecule.

For example, the VQE repeatedly executes the series of processes of setting the parameters of the quantum circuit, calculating the expected value of the ground energy by executing the quantum circuit, and calculating the parameters of the quantum circuit. The setting of first time is, for example, initializing the parameters of the quantum circuit. The setting of second time and beyond is, for example, setting the parameters of the quantum circuit calculated in the immediately-preceding series of processes. For example, the VQE is set in advance by the user.

The storage unit 400 stores, for example, information specifying the target molecule. The information specifying the target molecule is obtained by, for example, the obtaining unit 401.

The storage unit 400 stores, for example, a first quantum circuit for the case where the active space of the target molecule is reduced. The first quantum circuit is defined by, for example, the multiple first parameters. The first quantum circuit is obtained by, for example, the obtaining unit 401.

The storage unit 400 stores, for example, a second quantum circuit for the case where the active space of the target molecule is not reduced. The second quantum circuit is defined by, for example, the multiple second parameters. The number of the second parameters is larger than the number of the first parameters. The second quantum circuit is obtained by, for example, the obtaining unit 401.

The storage unit 400 stores, for example, the first value of each of the multiple first parameters of the first quantum circuit calculated first by the VQE for the case where the active space of the target molecule is reduced. The first value is calculated by, for example, the first execution unit 402. The storage unit 400 stores, for example, the second value of each of the multiple first parameters of the first quantum circuit calculated last by the VQE for the case where the active space of the target molecule is reduced. The second value is calculated by, for example, the first execution unit 402.

The storage unit 400 stores, for example, the third value of each of the multiple second parameters of the second quantum circuit calculated first by the VQE for the case where the active space of the target molecule is not reduced. For example, the third value is calculated first by the VQE by executing the VQE such that the parameters of the second quantum circuit are calculated only once. The third value is calculated by, for example, the second execution unit 403.

The obtaining unit 401 obtains various types of information used in the processing of each functional unit. The obtaining unit 401 stores the obtained various types of information in the storage unit 400, or outputs the obtained various types of information to each functional unit. The obtaining unit 401 may output the various types of information stored in the storage unit 400 to each functional unit. The obtaining unit 401 obtains the various types of information based on, for example, an operation input by the user. The obtaining unit 401 may receive the various types of information from, for example, an apparatus different from the information processing apparatus 100.

The obtaining unit 401 obtains, for example, the processing request demanding calculation of the expected value of the ground energy of the target molecule. For example, the obtaining unit 401 obtains the processing request by receiving the processing request from another computer. The another computer is, for example, the client apparatus 201. For example, the obtaining unit 401 may obtain the processing request by receiving an input of the processing request based on an operation input by the user.

The obtaining unit 401 obtains, for example, the information specifying the target molecule. The obtaining unit 401 obtains the information specifying the target molecule by, for example, receiving the information from another computer. The another computer is, for example, the client apparatus 201. The obtaining unit 401 may obtain the information specifying the target molecule by, for example, receiving an input of the information specifying the target molecule based on an operation input by the user. For example, when the information specifying the target molecule is included in the processing request, the obtaining unit 401 may obtain the information specifying the target molecule by extracting the information from the processing request.

The obtaining unit 401 obtains, for example, the first quantum circuit for the case where the active space of the target molecule is not reduced.

The obtaining unit 401 obtains the first quantum circuit by, for example, receiving the first quantum circuit from another computer. The another computer is, for example, the client apparatus 201. The obtaining unit 401 may obtain the first quantum circuit by, for example, receiving an input of the first quantum circuit based on an operation input by the user. For example, when the first quantum circuit is included in the processing request, the obtaining unit 401 may obtain the first quantum circuit by extracting the first quantum circuit from the processing request.

The obtaining unit 401 obtains, for example, the second quantum circuit for the case where the active space of the target molecule is not reduced. The obtaining unit 401 obtains the second quantum circuit by, for example, receiving the second quantum circuit from another computer. The another computer is, for example, the client apparatus 201. The obtaining unit 401 may obtain the second quantum circuit by, for example, receiving an input of the second quantum circuit based on an operation input by the user. For example, when the second quantum circuit is included in the processing request, the obtaining unit 401 may obtain the second quantum circuit by extracting the second quantum circuit from the processing request.

The obtaining unit 401 may receive a start trigger for starting processing of any of the functional units. The start trigger is, for example, presence of a predetermined operation input by the user. The start trigger may be, for example, reception of predetermined information from another computer. The start trigger may be, for example, output of predetermined information by any of the functional units.

The obtaining unit 401 receives, for example, the obtaining of the processing request as the start trigger for starting the processing of the first execution unit 402, the second execution unit 403, the setting unit 404, and the third execution unit 405.

The first execution unit 402 executes the VQE based on the first quantum circuit for the case where the active space of the target molecule is reduced. As a result of executing the VQE, the first execution unit 402 obtains the first value of each of the multiple first parameters of the first quantum circuit calculated first by the VQE. As a result of executing the VQE, the first execution unit 402 obtains the second value of each of the multiple first parameters of the first quantum circuit calculated last by the VQE. The first execution unit 402 may thereby obtain information to be the guideline for setting the initial value of each of the multiple second parameters.

The second execution unit 403 executes the VQE such that the parameters of the second quantum circuit are calculated once, based on the second quantum circuit for the case where the active space of the target molecule is not reduced. As a result of executing the VQE, the second execution unit 403 obtains the third value of each of the multiple second parameters of the second quantum circuit calculated first by the VQE. The second execution unit 403 may thereby obtain information to be the guideline for setting the initial value of each of the multiple second parameters.

The setting unit 404 specifies, for each of the multiple second parameters, one of the multiple first parameters for which the first value closest to the third value of the second parameter is calculated, based on the calculated first value and the calculated third value, to associate the specified first parameter with the second parameter. The setting unit 404 may thereby specify the first parameter similar to the second parameter, and may specify the first parameter preferable to be referred to in setting of the initial value of the second parameter.

When the VQE is to be executed for the case where the active space of the target molecule is not reduced, the setting unit 404 sets the initial value of each of the multiple second parameters to the second value of one of the multiple first parameters specified to be associated with the second parameter. The setting unit 404 may thereby set the initial value of each of the multiple second parameters to an appropriate value. The setting unit 404 may thus reduce the processing time taken to execute the VQE for the case where the active space of the target molecule is not reduced.

The third execution unit 405 executes the VQE for the case where the active space of the target molecule is not reduced, based on the second quantum circuit and the initial value of each of the multiple second parameters set by the setting unit 404. As a result of executing the VQE, the third execution unit 405 calculates the expected value of the ground energy of the target molecule. The third execution unit 405 may thereby facilitate analysis of the properties and the like of the target molecule by the user.

The output unit 406 outputs a processing result of at least one of the functional units. An output form is, for example, displaying the processing result on a display, outputting the processing result to a printer for printing, transmitting the processing result to an external apparatus through the network I/F 303, or storing the processing result in the storage area of the memory 302, the recording medium 305, or the like. The output unit 406 may thereby notify the user of the processing result of at least one of the functional units, and achieve an improvement in convenience of the information processing apparatus 100.

The output unit 406 outputs, for example, the initial value of each of the multiple second parameters set by the setting unit 404. For example, the output unit 406 outputs the initial value of each of the multiple second parameters set by the setting unit 404 such that it is possible for the user to refer to the initial value. For example, the output unit 406 transmits the initial value of each of the multiple second parameters set by the setting unit 404 to another computer. The another computer is, for example, the client apparatus 201. The output unit 406 may thereby enable usage of the initial value of each of the multiple second parameters set by the setting unit 404 outside the information processing apparatus 100.

The output unit 406 outputs, for example, the expected value of the ground energy of the target molecule calculated by the third execution unit 405. For example, the output unit 406 outputs the expected value of the ground energy of the target molecule calculated by the third execution unit 405 such that it is possible for the user to refer to the expected value. For example, the output unit 406 transmits the expected value of the ground energy of the target molecule calculated by the third execution unit 405 to another computer. The another computer is, for example, the client apparatus 201. The output unit 406 may thereby enable usage of the expected value of the ground energy of the target molecule calculated by the third execution unit 405 outside the information processing apparatus 100.

Although the case where the information processing apparatus 100 includes the obtaining unit 401, the first execution unit 402, the second execution unit 403, the setting unit 404, and the third execution unit 405 has been explained, the embodiment is not limited to this case. For example, there may be a case where the information processing apparatus 100 does not include one or more of the functional units. For example, there may be a case where the information processing apparatus 100 does not include the third execution unit 405. For example, there may be a case where the information processing apparatus 100 transmits the initial value set by the setting unit 404 to another computer including the third execution unit 405.

### Operation Example of Information Processing Apparatus 100

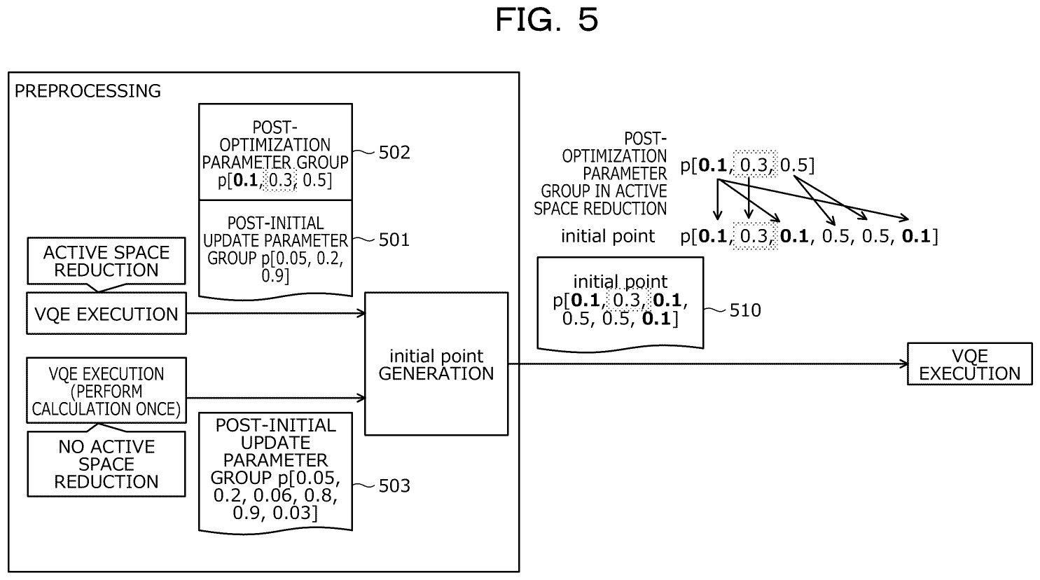

Next, an operation example of the information processing apparatus 100 will be explained with reference to FIGS. 5 to 7.

FIGS. 5 to 7 are explanatory diagrams illustrating the operation example of the information processing apparatus 100. In FIG. 5, (5-1) the information processing apparatus 100 specifies the first quantum circuit corresponding to the target molecule, for the case where the active space of the target molecule is reduced. The first quantum circuit is defined by values of n parameters. Here, n is the number of parameters. The n parameters of the first quantum circuit have, respectively, indices i provided sequentially from the first parameter. The indices i are 1, 2, . . . , n.

The information processing apparatus 100 executes the VQE based on the specified first quantum circuit for the case where the active space of the target molecule is reduced, until a predetermined convergence condition is satisfied. As a result of executing the VQE, the information processing apparatus 100 obtains a post-initial update parameter group 501=p[0.05, 0.2, 0.9] of the first quantum circuit calculated first by the VQE. In the following explanation, the parameter with the index i in the post-initial update parameter group 501 is referred to as “s[i]” in some cases. As a result of executing the VQE, the information processing apparatus 100 obtains a post-optimization parameter group 502=p[0.1, 0.3, 0.5] of the first quantum circuit calculated last by the VQE. In the following explanation, the value of the parameter with the index i in the post-optimization parameter group 502 is referred to as “o[i]” in some cases.

(5-2) The information processing apparatus 100 specifies the second quantum circuit corresponding to the target molecule for the case where the active space of the target molecule is not reduced. The second quantum circuit is defined by values of m parameters. Here, m is the number of parameters. Here, m is a value larger than n. The m parameters of the second quantum circuit have, respectively, indices j provided sequentially from the first parameter. The indices j are 1, 2, . . . , m.

The information processing apparatus 100 executes the VQE such that the parameters of the second quantum circuit are calculated only once, based on the specified second quantum circuit for the case where the active space of the target molecule is not reduced. As a result of executing the VQE, the information processing apparatus 100 obtains a post-initial update parameter group 503=p[0.05, 0.2, 0.06, 0.8, 0.9, 0.03] of the second quantum circuit calculated first by the VQE. In the following explanation, the value of the parameter with the index j in the post-initial update parameter group 503 is referred to as “l[j]” in some cases.

(5-3) The information processing apparatus 100 generates an initial value group initial_point 510 related to the parameters of the second quantum circuit, based on the post-initial update parameter group 501, the post-optimization parameter group 502, and the post-initial update parameter group 503. For example, for the index j of each of the m parameters of the second quantum circuit, the information processing apparatus 100 specifies the index i of one of the n parameters of the first quantum circuit for which |l[j]-s[i]| is the smallest. For example, the information processing apparatus 100 stores a correspondence relationship between the index j and the index i specified for the index j.

For example, the information processing apparatus 100 sets the parameter o[i] with the index i corresponding to the index j in the post-optimization parameter group 502, as the initial value of the parameter of the second quantum circuit with the index j, based on the stored correspondence relationship. For example, the information processing apparatus 100 generates initial_point 510 in which the set initial value of each of the m parameters of the second quantum circuit is recorded. The information processing apparatus 100 generates, for example, initial_point 510=p[0.1, 0.3, 0.1, 0.5, 0.5, 0.1].

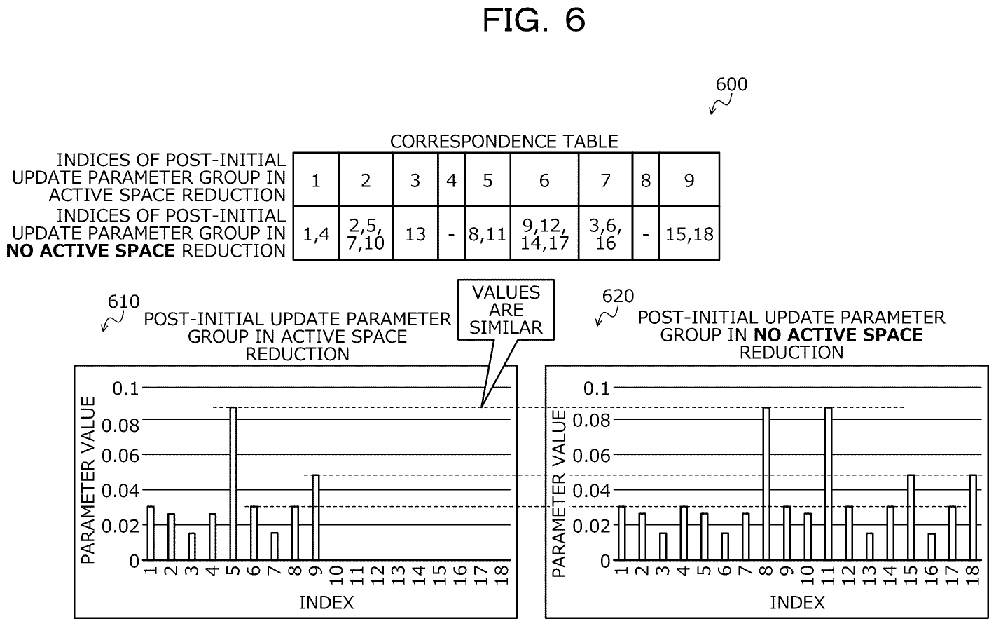

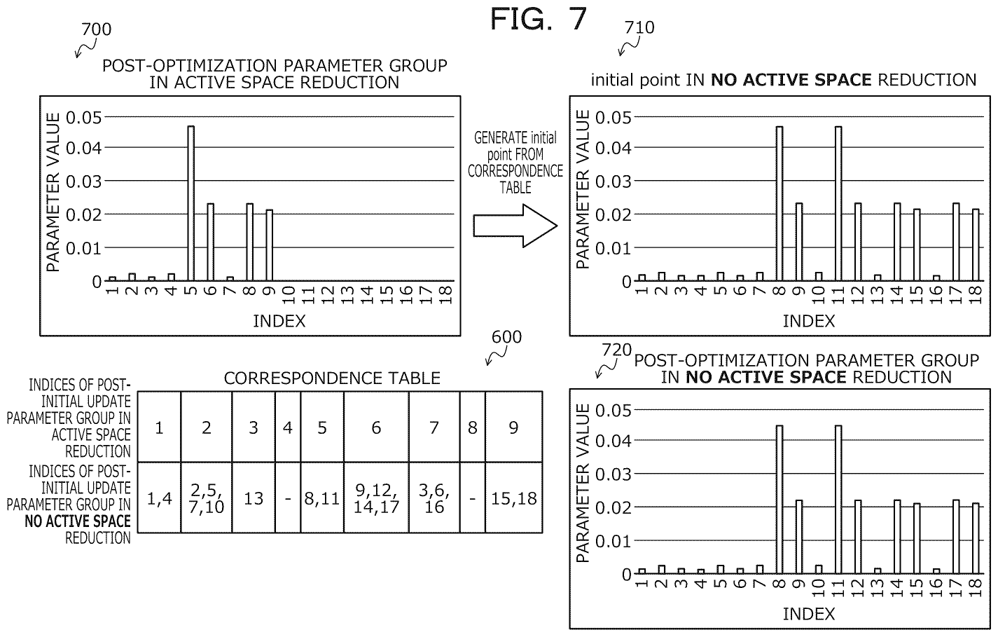

(5-4) The information processing apparatus 100 sets the initial value of each of the m parameters of the second quantum circuit based on initial_point 510, and executes the VQE for the case where the active space of the target molecule is not reduced. The information processing apparatus 100 may thereby set the initial value of each of the m parameters of the second quantum circuit to an appropriate value. The information processing apparatus 100 may thus reduce the processing time taken to execute the VQE for the case where the active space of the target molecule is not reduced. Next, the description proceeds to explanation of FIGS. 6 and 7, and explanation is given of a specific example in which the information processing apparatus 100 generates initial_point 510 and sets the initial value of each of the m parameters of the second quantum circuit.

In FIG. 6, assume that the information processing apparatus 100 has obtained the post-initial update parameter group 501 illustrated in the graph 610. Assume that the information processing apparatus 100 has obtained the post-initial update parameter group 503 illustrated in the graph 620. For the index j of each of the m parameters of the second quantum circuit, the information processing apparatus 100 specifies the index i of one of the n parameters of the first quantum circuit having a value similar to a value of the parameter with the index j.

For example, for the index j of each of the m parameters of the second quantum circuit, the information processing apparatus 100 specifies the index i of one of the n parameters of the first quantum circuit for which |l[j]-s[i]| is the smallest. For example, the information processing apparatus 100 stores a correspondence relationship between the index j and the index i specified for the index j in the correspondence table 600.

In the example of FIG. 6, the information processing apparatus 100 specifies the index i=1 of the first parameter of the first quantum circuit, for the index j=1 of the first parameter of the second quantum circuit, as illustrated in the correspondence table 600. The information processing apparatus 100 specifies the index i=2 of the second parameter of the first quantum circuit, for the index j=2 of the second parameter of the second quantum circuit, as illustrated in the correspondence table 600. The information processing apparatus 100 specifies the index i=7 of the seventh parameter of the first quantum circuit, for the index j=3 of the third parameter of the second quantum circuit, as illustrated in the correspondence table 600.

The information processing apparatus 100 specifies the index i=1 of the first parameter of the first quantum circuit, for the index j=4 of the fourth parameter of the second quantum circuit, as illustrated in the correspondence table 600. The information processing apparatus 100 specifies the index i=2 of the second parameter of the first quantum circuit, for the index j=5 of the fifth parameter of the second quantum circuit, as illustrated in the correspondence table 600. The information processing apparatus 100 specifies the index i=7 of the seventh parameter of the first quantum circuit, for the index j=6 of the sixth parameter of the second quantum circuit, as illustrated in the correspondence table 600.

The information processing apparatus 100 specifies the index i=2 of the second parameter of the first quantum circuit, for the index j=7 of the seventh parameter of the second quantum circuit, as illustrated in the correspondence table 600. The information processing apparatus 100 specifies the index i=5 of the fifth parameter of the first quantum circuit, for the index j=8 of the eighth parameter of the second quantum circuit, as illustrated in the correspondence table 600. The information processing apparatus 100 specifies the index i=6 of the sixth parameter of the first quantum circuit, for the index j=9 of the ninth parameter of the second quantum circuit, as illustrated in the correspondence table 600.

The information processing apparatus 100 specifies the index i=2 of the second parameter of the first quantum circuit, for the index j=10 of the tenth parameter of the second quantum circuit, as illustrated in the correspondence table 600. The information processing apparatus 100 specifies the index i=5 of the fifth parameter of the first quantum circuit, for the index j=11 of the eleventh parameter of the second quantum circuit, as illustrated in the correspondence table 600. The information processing apparatus 100 specifies the index i=6 of the sixth parameter of the first quantum circuit, for the index j=12 of the twelfth parameter of the second quantum circuit, as illustrated in the correspondence table 600.

The information processing apparatus 100 specifies the index i=3 of the third parameter of the first quantum circuit, for the index j=13 of the thirteenth parameter of the second quantum circuit, as illustrated in the correspondence table 600. The information processing apparatus 100 specifies the index i=6 of the sixth parameter of the first quantum circuit, for the index j=14 of the fourteenth parameter of the second quantum circuit, as illustrated in the correspondence table 600. The information processing apparatus 100 specifies the index i=9 of the ninth parameter of the first quantum circuit, for the index j=15 of the fifteenth parameter of the second quantum circuit, as illustrated in the correspondence table 600.

The information processing apparatus 100 specifies the index i=7 of the seventh parameter of the first quantum circuit, for the index j=16 of the sixteenth parameter of the second quantum circuit, as illustrated in the correspondence table 600. The information processing apparatus 100 specifies the index i=6 of the sixth parameter of the first quantum circuit, for the index j=17 of the seventeenth parameter of the second quantum circuit, as illustrated in the correspondence table 600. The information processing apparatus 100 specifies the index i=9 of the ninth parameter of the first quantum circuit, for the index j=18 of the eighteenth parameter of the second quantum circuit, as illustrated in the correspondence table 600. Next, the description proceeds to explanation of FIG. 7.

In FIG. 7, assume that the information processing apparatus 100 has obtained the post-optimization parameter group 502 illustrated in the graph 700. For example, the information processing apparatus 100 sets the initial value of the parameter of the second quantum circuit with the index j to the parameter o[i] with the index i corresponding to the index j in the post-optimization parameter group 502, based on the correspondence table 600. The information processing apparatus 100 generates initial_point 510 illustrated in the graph 710 in which the set initial value of each of the m parameters of the second quantum circuit is recorded.

In the example of FIG. 7, the information processing apparatus 100 sets the value o[1] of the parameter with the index i=1 in the post-optimization parameter group 502, as the initial value of the first parameter of the second quantum circuit with the index j=1. The information processing apparatus 100 specifies the value o[2] of the parameter with the index i=2 in the post-optimization parameter group 502, as the initial value of the second parameter of the second quantum circuit with the index j=2. The information processing apparatus 100 specifies the value o[7] of the parameter with the index i=7 in the post-optimization parameter group 502, as the initial value of the third parameter of the second quantum circuit with the index j=3.

The information processing apparatus 100 specifies the value o[1] of the parameter with the index i=1 in the post-optimization parameter group 502, as the initial value of the fourth parameter of the second quantum circuit with the index j=4. The information processing apparatus 100 specifies the value o[2] of the parameter with the index i=2 in the post-optimization parameter group 502, as the initial value of the fifth parameter of the second quantum circuit with the index j=5. The information processing apparatus 100 specifies the value o[7] of the parameter with the index i=7 in the post-optimization parameter group 502, as the initial value of the sixth parameter of the second quantum circuit with the index j=6.

The information processing apparatus 100 specifies the value o[2] of the parameter with the index i=2 in the post-optimization parameter group 502, as the initial value of the seventh parameter of the second quantum circuit with the index j=7. The information processing apparatus 100 specifies the value o[5] of the parameter with the index i=5 in the post-optimization parameter group 502, as the initial value of the eighth parameter of the second quantum circuit with the index j=8. The information processing apparatus 100 specifies the value o[6] of the parameter with the index i=6 in the post-optimization parameter group 502, as the initial value of the ninth parameter of the second quantum circuit with the index j=9.

The information processing apparatus 100 specifies the value o[2] of the parameter with the index i=2 in the post-optimization parameter group 502, as the initial value of the tenth parameter of the second quantum circuit with the index j=10. The information processing apparatus 100 specifies the value o[5] of the parameter with the index i=5 in the post-optimization parameter group 502, as the initial value of the eleventh parameter of the second quantum circuit with the index j=11. The information processing apparatus 100 specifies the value o[6] of the parameter with the index i=6 in the post-optimization parameter group 502, as the initial value of the twelfth parameter of the second quantum circuit with the index j=12.

The information processing apparatus 100 specifies the value o[3] of the parameter with the index i=3 in the post-optimization parameter group 502, as the initial value of the thirteenth parameter of the second quantum circuit with the index j=13. The information processing apparatus 100 specifies the value o[6] of the parameter with the index i=6 in the post-optimization parameter group 502, as the initial value of the fourteenth parameter of the second quantum circuit with the index j=14. The information processing apparatus 100 specifies the value o[9] of the parameter with the index i=9 in the post-optimization parameter group 502, as the initial value of the fifteenth parameter of the second quantum circuit with the index j=15.

The information processing apparatus 100 specifies the value o[7] of the parameter with the index i=7 in the post-optimization parameter group 502, as the initial value of the sixteenth parameter of the second quantum circuit with the index j=16. The information processing apparatus 100 specifies the value o[6] of the parameter with the index i=6 in the post-optimization parameter group 502, as the initial value of the seventeenth parameter of the second quantum circuit with the index j=17. The information processing apparatus 100 specifies the value o[9] of the parameter with the index i=9 in the post-optimization parameter group 502, as the initial value of the eighteenth parameter of the second quantum circuit with the index j=18.

The information processing apparatus 100 sets the initial value of each of the m parameters of the second quantum circuit based on initial_point 510, and executes the VQE for the case where the active space of the target molecule is not reduced. A post-optimization parameter group of the second quantum circuit calculated last by the VQE as a result of the information processing apparatus 100 executing the VQE is illustrated in the graph 720. As illustrated in the graphs 710 and 720, initial_point 510 tends to be similar to, for example, the post-optimization parameter group of the second quantum circuit. Thus, initial_point 510 is assumed to express an initial value group that is related to the parameters of the second quantum circuit and that is suitable for the purpose of reducing the processing time taken to execute the VQE.

The information processing apparatus 100 may thereby set the initial value of each of the m parameters of the second quantum circuit to an appropriate value. The information processing apparatus 100 may thus reduce the processing time taken to execute the VQE for the case where the active space of the target molecule is not reduced.

For example, as preparation for generating initial_point 510, the information processing apparatus 100 may reduce the active space of the target molecule, and then execute the VQE. For example, as preparation for generating initial_point 510, the information processing apparatus 100 may execute the VQE such that the parameters of the quantum circuit are calculated only once, for the case where the active space of the target molecule is not reduced. The information processing apparatus 100 may thus reduce time taken for preparation.

Accordingly, for example, the information processing apparatus 100 may reduce time taken to generate initial_point 510. For example, since the information processing apparatus 100 may execute the VQE based on appropriate initial_point 510 for the case where the active space of the target molecule is not reduced, the information processing apparatus 100 may reduce the processing time taken to execute the VQE.

Assume that processing time per one step of the VQE in the case where the active space of the target molecule is not reduced is x. Meanwhile, assume that processing time per one step of the VQE in the case where the active space of the target molecule is reduced is y. Here, x>>y is satisfied.

Assume that the number of steps of the VQE in the case where the initial value of each of the m parameters of the second quantum circuit is set to an inappropriate value for the case where the active space of the target molecule is not reduced is a. Meanwhile, assume that the number of steps of the VQE in the case where the initial value of each of the m parameters of the second quantum circuit is set to an appropriate value for the case where the active space of the target molecule is not reduced is b. Here, a>>b is satisfied.

Assume that the number of steps of the VQE in the case where the initial value of each of the n parameters of the first quantum circuit is set to an inappropriate value for the case where the active space of the target molecule is reduced is c. Here, c>>b is satisfied. Here, c≈a is satisfied.

According to a method of a related art, for example, the VQE is sometimes executed with the initial value of each of the m parameters of the second quantum circuit set to an inappropriate value for the case where the active space of the target molecule is not reduced. Thus, in the method of related art, processing time T1 from the start of execution of the VQE to the end of execution of the VQE is a×x.

Meanwhile, the information processing apparatus 100 may execute the VQE while setting the initial value of each of the m parameters of the second quantum circuit to an appropriate value for the case where the active space of the target molecule is not reduced. Processing time T2 from the start of execution of the VQE to the end of execution of the VQE in the information processing apparatus 100 is c×y+b×x. If x>>y, a>>b, and c≈a are satisfied, T1>>T2 tends to be satisfied. The information processing apparatus 100 may thus reduce the processing time taken to execute the VQE for the case where the active space of the target molecule is not reduced.

### Overall Processing Procedure

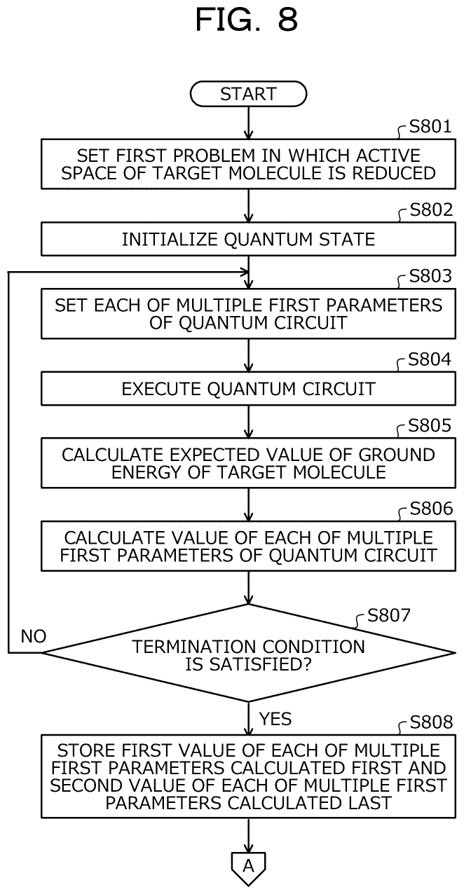

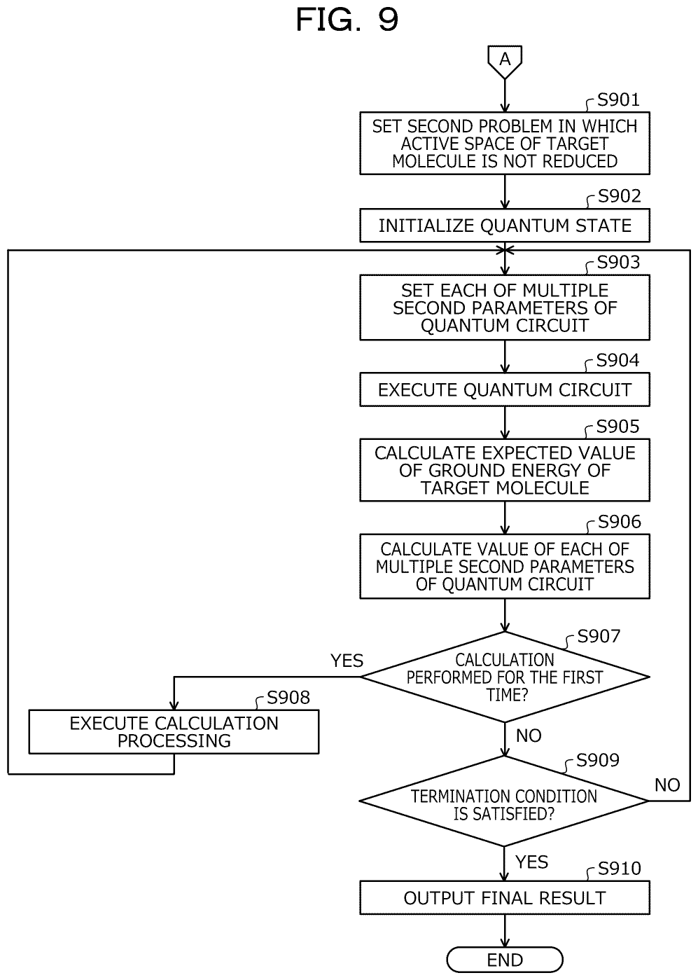

Next, an example of an overall processing procedure executed by the information processing apparatus 100 will be explained by using FIGS. 8 and 9. The overall processing is implemented by, for example, the CPU 301, the storage areas of the memory 302, the recording medium 305, and the like, and the network I/F 303 illustrated in FIG. 3.

FIGS. 8 and 9 are flowcharts illustrating the example of the overall processing procedure. In FIG. 8, the information processing apparatus 100 sets a first problem in which the active space of the target molecule is reduced (step S801). The information processing apparatus 100 initializes a quantum state for the first problem (step S802).

The information processing apparatus 100 sets each of the multiple first parameters of the quantum circuit for the first problem (step S803). The information processing apparatus 100 executes the quantum circuit for the first problem (step S804). The information processing apparatus 100 calculates the expected value of the ground energy of the target molecule for the first problem (step S805). The information processing apparatus 100 calculates the value of each of the multiple first parameters of the quantum circuit based on the calculated expected value for the first problem (step S806).

The information processing apparatus 100 determines whether or not a termination condition is satisfied (step S807). When the termination condition is not satisfied (step S807: No), the information processing apparatus 100 returns to the processing of step S803. Meanwhile, when the termination condition is satisfied (step S807: Yes), the information processing apparatus 100 proceeds to processing of step S808.

In step S808, the information processing apparatus 100 stores the first value of each of the multiple first parameters calculated first and the second value of each of the multiple first parameters calculated last (step S808). The information processing apparatus 100 proceeds to processing of step S901 in FIG. 9.

In FIG. 9, the information processing apparatus 100 sets a second problem in which the active space of the target molecule is not reduced (step S901). The information processing apparatus 100 initializes the quantum state for the second problem (step S902).

The information processing apparatus 100 sets each of the multiple second parameters of the quantum circuit for the second problem (step S903). The information processing apparatus 100 executes the quantum circuit for the second problem (step S904). The information processing apparatus 100 calculates the expected value of the ground energy of the target molecule for the second problem (step S905). The information processing apparatus 100 calculates the value of each of the multiple second parameters of the quantum circuit based on the calculated expected value for the second problem (step S906).

The information processing apparatus 100 determines whether or not the calculation is calculation performed for the first time (step S907). When the calculation is calculation performed for the first time (step S907: Yes), the information processing apparatus 100 proceeds to processing of step S908. Meanwhile, when the calculation is not the calculation performed for the first time (step S907: No), the information processing apparatus 100 proceeds to processing of step S909.

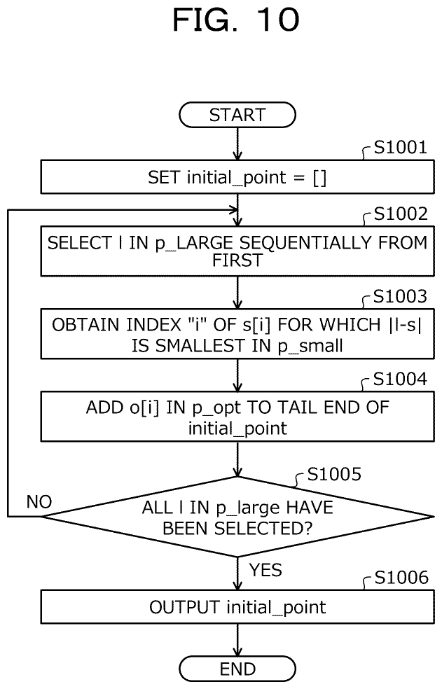

In step S908, the information processing apparatus 100 executes calculation processing to be described later in FIG. 10 (step S908). The information processing apparatus 100 returns to the processing of step S903.

In step S909, the information processing apparatus 100 determines whether or not a termination condition is satisfied (step S909). When the termination condition is not satisfied (step S909: No), the information processing apparatus 100 returns to the processing of step S903. Meanwhile, when the termination condition is satisfied (step S909: Yes), the information processing apparatus 100 proceeds to processing of step S910.

In step S910, the information processing apparatus 100 outputs a final result (step S910). The information processing apparatus 100 terminates the overall processing.

### Calculation Processing Procedure

Next, an example of a calculation processing procedure executed by the information processing apparatus 100 will be explained by using FIG. 10. The calculation processing is implemented by, for example, the CPU 301, the storage areas of the memory 302, the recording medium 305, and the like, and the network I/F 303 illustrated in FIG. 3.

FIG. 10 is a flowchart illustrating an example of a calculation processing procedure. In FIG. 10, the information processing apparatus 100 sets initial_point=[] (step S1001). The information processing apparatus 100 sets a list in which the value of each of the multiple first parameters o calculated last for the case where the active space is reduced is recorded, as p_opt. In the following explanation, the parameter o with the index i is referred to as “o[i]” in some cases.

The information processing apparatus 100 sets a list in which the value of each of the multiple first parameters s calculated first for the case where the active space is reduced is recorded, as p_small. In the following explanation, the parameter s with the index i is referred to as “s[i]” in some cases. The information processing apparatus 100 sets a list in which the value of each of the multiple second parameters l calculated first for the case where the active space is not reduced is recorded, as p_large. In the following explanation, the parameter l with the index i is referred to as “l[i]” in some cases.

The information processing apparatus 100 selects l of p_large sequentially from the first l (step S1002). The information processing apparatus 100 obtains the index “i” of s[i] for which |l-s| is the smallest in p_small (step S1003). The information processing apparatus 100 adds o [i] in p_opt to the tail end of initial_point (step S1004).

The information processing apparatus 100 determines whether or not all l in p_large have been selected (step S1005). When an unselected l is present (step S1005: No), the information processing apparatus 100 returns to the processing of step S1002. Meanwhile, when all l in p_large have been selected (step S1005: Yes), the information processing apparatus 100 proceeds to processing of step S1006.

In step S1006, the information processing apparatus 100 outputs initial_point (step S1006). The information processing apparatus 100 terminates the calculation processing.

The information processing apparatus 100 may execute the processes of the steps in each of the flowcharts illustrated in FIGS. 8 to 10 while changing the processing order of some of the steps. The information processing apparatus 100 may skip the processes of some of the steps in each of the flowcharts illustrated in FIGS. 8 to 10.

As described above, according to the information processing apparatus 100, it is possible to execute the VQE for the case where the active space of the target molecule is reduced. According to the information processing apparatus 100, it is possible to obtain the first value of each of the multiple first parameters calculated first by the VQE and the second value of each of the multiple first parameters calculated last by the VQE. According to the information processing apparatus 100, it is possible to execute the VQE such that the parameters of the quantum circuit are calculated once, for the case where the active space of the target molecule is not reduced. According to the information processing apparatus 100, it is possible to obtain the third value of each of the multiple second parameters calculated first by the VQE, the number of the multiple second parameters being larger than the number of the multiple first parameters. According to the information processing apparatus 100, it is possible to specify, for each of the multiple second parameters, one of the multiple first parameters for which the first value closest to the third value of the second parameter is calculated, to associate the specified first parameter with the second parameter. According to the information processing apparatus 100, when the VQE is to be executed for the case where the active space of the target molecule is not reduced, it is possible to set the initial value of each of the multiple second parameters to the second value of one of the first parameters specified to be associated with the second parameter. The information processing apparatus 100 may thereby reduce the processing time taken to execute the VQE for the case where the active space of the target molecule is not reduced.

According to the information processing apparatus 100, it is possible to execute the VQE for the case where the active space of the target molecule is not reduced, based on the set initial value of each of the multiple second parameters. The information processing apparatus 100 may thereby execute the VQE for the case where the active space of the target molecule is not reduced in itself.

According to the information processing apparatus 100, it is possible to calculate the expected value of the ground energy of the target molecule by executing the VQE for the case where the active space of the target molecule is not reduced, based on the set initial value of each of the multiple second parameters. The information processing apparatus 100 may thereby facilitate analysis of the properties of the target molecule.

The information processing method explained in the present embodiment may be achieved by executing a pre-prepared program in a computer such as a PC or a workstation. The information processing program explained in the present embodiment is recorded in a computer-readable recording medium, and is executed by being read from the recording medium by a computer. The recording medium is a hard disk, a flexible disk, a compact disc (CD)-ROM, a magneto optical (MO) disc, a Digital Versatile Disc (DVD), or the like. The information processing program described in the present embodiment may be distributed via a network such as the Internet.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing an information processing program that causes a computer to execute processing of:
obtaining a first value of each of a plurality of first parameters calculated first by the variational quantum eigensolver that recurrently calculates values of parameters of a quantum circuit and a second value of each of the plurality of first parameters calculated last by the variational quantum eigensolver by executing the variational quantum eigensolver for a case where an active space of a target molecule is reduced;
obtaining a third value of each of a plurality of second parameters calculated first by the variational quantum eigensolver by executing the variational quantum eigensolver such that the parameters of the quantum circuit are calculated once, for a case where the active space of the target molecule is not reduced;
specifying, for each of the plurality of second parameters, one of the first parameters that corresponds to the first value closest to the third value of the second parameter, based on the calculated first value and the calculated third value, to associate the specified first parameter with the second parameter; and
when the variational quantum eigensolver is to be executed for the case where the active space of the target molecule is not reduced, setting an initial value of each of the plurality of second parameters to the second value of the one of the plurality of first parameters specified to be associated with the each of the plurality of second parameters.

2. The non-transitory computer-readable recording medium according to claim 1 that causes the computer to execute processing of executing the variational quantum eigensolver for the case where the active space of the target molecule is not reduced, based on the set initial value of each of the plurality of second parameters.

3. The non-transitory computer-readable recording medium according to claim 1, wherein the number of the plurality of second parameters is larger than the number of the plurality of first parameters.

4. The non-transitory computer-readable recording medium according to claim 2, wherein the executed processing is calculating an expected value of ground energy of the target molecule by executing the variational quantum eigensolver for the case where the active space of the target molecule is not reduced, based on the set initial value of each of the plurality of second parameters.

5. A information processing method that causes a computer to execute processing of:
obtaining a first value of each of a plurality of first parameters calculated first by the variational quantum eigensolver that recurrently calculates values of parameters of a quantum circuit and a second value of each of the plurality of first parameters calculated last by the variational quantum eigensolver by executing the variational quantum eigensolver for a case where an active space of a target molecule is reduced;
obtaining a third value of each of a plurality of second parameters calculated first by the variational quantum eigensolver by executing the variational quantum eigensolver such that the parameters of the quantum circuit are calculated once, for a case where the active space of the target molecule is not reduced;
specifying, for each of the plurality of second parameters, one of the first parameters that corresponds to the first value closest to the third value of the second parameter, based on the calculated first value and the calculated third value, to associate the specified first parameter with the second parameter; and
when the variational quantum eigensolver is to be executed for the case where the active space of the target molecule is not reduced, setting an initial value of each of the plurality of second parameters to the second value of the one of the plurality of first parameters specified to be associated with the each of the plurality of second parameters.

6. A information processing apparatus comprising:
a memory; and
a process coupled to the memory and configured to:
obtain a first value of each of a plurality of first parameters calculated first by the variational quantum eigensolver that recurrently calculates values of parameters of a quantum circuit and a second value of each of the plurality of first parameters calculated last by the variational quantum eigensolver by executing the variational quantum eigensolver for a case where an active space of a target molecule is reduced;
obtain a third value of each of a plurality of second parameters calculated first by the variational quantum eigensolver by executing the variational quantum eigensolver such that the parameters of the quantum circuit are calculated once, for a case where the active space of the target molecule is not reduced;
specify, for each of the plurality of second parameters, one of the first parameters that corresponds to the first value closest to the third value of the second parameter, based on the calculated first value and the calculated third value, to associate the specified first parameter with the second parameter; and
when the variational quantum eigensolver is to be executed for the case where the active space of the target molecule is not reduced, set an initial value of each of the plurality of second parameters to the second value of the one of the plurality of first parameters specified to be associated with the each of the plurality of second parameters.
